(19) United States
(12) Patent Application Publication
(10) Pub. No.: US 2026/0348957 A1
(43) Pub. Date: Nov. 5, 2026

(54) METHOD AND KIT FOR SCREENING HEAT-RESISTANT PROTEINS

(71) Applicant: NATIONAL UNIVERSITY CORPORATION TOTTORI UNIVERSITY, Tottori-Shi, Tottori (JP)

(72) Inventor: Suzuki Hirokazu, Tottori-Shi, Tottori (JP)

(21) Appl. No.: 19/131,492

(22) PCT Filed: Jan. 25, 2024
(86) PCT No.: PCT/JP2024/002137

§ 371 Date: May 20, 2025

(30) Foreign Application Priority Data
Nov. 30, 2022 (JP) 2022-191608

(51) Int. Cl. G01N 33/569; C12N 15/75

(52) U.S. Cl. CPC G01N 33/56911; C12N 15/75; G01N 2333/32

(57) ABSTRACT

A method and a kit for screening for a thermostable polypeptide are provided, the method comprising: a step of culturing, at a screening temperature of 45 °C or higher, a population of thermophile transformed with a nucleic acid library comprising multiple nucleic acids encoding multiple target polypeptides, the multiple target polypeptides having differences in amino acid sequences and each having, at a terminus, a heterologous peptide of a common sequence wherein the heterologous peptide is shorter than 50 amino acids; a step of adding a reporter polypeptide, which binds to the heterologous peptide, to a sample of multiple clones derived from the population of thermophile; a step of detecting the amount of binding of the reporter polypeptide and identifying one or more clones having an elevated amount of binding as clones of the thermophile which have accumulated thermostable target polypeptides which are resistant to the screening temperature.

### TECHNICAL FIELD

The present disclosure relates to the field of developing and producing proteins using genetic engineering technology.

### BACKGROUND ART

Artificial proteins produced by genetic engineering technology have large potential for industrial applications, and in fact they have been already widely used in various industrial sectors including pharmaceutical, catalyst, and materials industries. One of the factors which often hinder development, production, and utilization of industrially useful artificial proteins is instability of such artificial proteins, especially their instability to heat. For example, proteins (enzymes) utilized as catalysts for industrial reactions are required to be stable at the temperatures at which the reactions take place (which are often higher than room temperature or biological temperature), but proteins which are stable at such temperatures and able to provide the needed enzymatic activities are rare in nature in the first place, and developing such proteins artificially based on natural proteins is also not an easy task. There is usually no clue as to which amino acid residues should be mutated for the improvement of thermostability, and the overwhelming majority of mutations not only fail to bring improvement of thermostability but also are deleterious to the proper folding and functioning of the proteins. Furthermore, how to identify the very rare mutant proteins which may have improved thermostability is also a difficult problem. For example, clones in a randomly mutated library may be isolated one by one to analyze the protein expression, in the hope of finding a thermostable one from the individual protein analyses, but such an approach would be extremely inefficient.

Non-Patent Document 1 discloses introducing randomly mutagenized kanamycin nucleotidyl transferase genes into the thermophile Bacillus stearothermophilus (now re-classified as Geobacillus stearothermophilus), culturing them at a high temperature and in the presence of kanamycin, and selecting kanamycin-resistant clones to identify the mutant enzymes having increased thermostability. This screening method relies on the enzymatic activity of the mutated proteins themselves in order to identify the thermostable mutant variants. Non-Patent Document 2 provides a similar example which identified thermostable mutant variants, capable of conferring viability on the host bacteria under a selective pressure, by relying on the enzymatic activity of the mutated proteins themselves.

In contrast, Non-Patent Document 3 discloses a screening experiment in which a randomly mutagenized target protein was fused to the thermostable kanamycin nucleotidyl transferase of Non-Patent Document 1 (as a reporter) and expressed in the thermophile Thermus thermophilus cultured at a high temperature and in the presence of kanamycin, to identify the clones, which retained the enzymatic reporter activity, as expressing thermostable target proteins. This screening method was based on the principle that a target protein that was thermally unstable and therefore not folded properly would also adversely affect the higher order folding of the enzymatic reporter to which it was fused. This method could advantageously identify thermostable mutant variants independent of any activity of the mutagenized protein itself. However, the necessity of fusing the target protein to the reporter protein, which had a relatively large size of over 250 amino acids, could present a limitation.

Non-Patent Document 4 discloses a method comprising providing Escherichia coli colonies which have overexpressed target proteins on a special filter membrane and exposing the E. coli colonies to an increased temperature, and then lysing the cells. This method is based on the principle that the target protein which is thermally unstable and therefore results in unfolding followed by aggregation would be retained on the filter membrane whereas the target protein which could remain soluble even at the increased temperature would pass through the filter membrane and be captured for the detection on the nitrocellulose layer disposed under the filter membrane. This method relies on the use of the special filter structures.

Thermostable proteins have the general tendency to exhibit robustness not only under the heat but also under other environments, and therefore methods for identifying thermostable proteins would be industrially highly valuable. Furthermore, thermostable enzymes may have other advantages such as higher activity even at room temperature, ease of storage, and longer shelf lives. Moreover, identification of thermostable proteins may lead to, for example, understanding of positions and types of amino acid residues conferring the thermostability, which could in turn provide deeper understanding of structural biology of the proteins. Several ingenious methods have been developed to screen for, and identify, thermostable proteins with improved efficiency, as described above, but they are still few and each have their own unique limitations. In any case, it still remains generally true that screening for and identifying thermostable proteins presents a difficult problem.

### CITATION LIST

### Non-Patent Documents

Non-Patent Document 1: J. Biol. Chem. (1985) 260:15298-303
Non-Patent Document 2: Appl. Environ. Microbiol. (2015) 81:149-58.
Non-Patent Document 3: Nat. Methods (2007) 4:919-21
Non-Patent Document 4: Nat. Commun. (2013) 4:2901

### SUMMARY OF THE INVENTION

### Problem to be Solved by the Invention

An objective of the embodiments of the present disclosure is the provision of a novel technique for screening for thermostable proteins which at least partially overcomes the limitations of the prior arts. In particular, an objective is the provision of a novel screening method which does not need to rely on enzymatic activity of the target proteins themselves to identify those which are thermostable.

### Solution to the Problem

In the course of researches attempting to express various heterologous proteins in thermophiles growing under high temperatures, the present inventor has noticed a phenomenon in which, while thermostable proteins accumulate in the samples, non-thermostable proteins not just turn into denatured or misfolded forms but also fail to be detected even in such misfolded forms and they appear to not accumulate substantially in the first place. This is a very simple rule but has thus far escaped conscious appreciation. The inventor has sought to develop screening methods on the basis of the hypothesis that thermostable proteins could be screened for by assuming this rule, and has indeed obtained observations and results consistent with the hypothesis, which has led to the inventions disclosed herein.

The present disclosure includes the following embodiments.

[1] A method of screening for a thermostable polypeptide, the method comprising:
a step of culturing, at a screening temperature of 45 °C or higher, a population of thermophile transformed with a nucleic acid library comprising multiple nucleic acids encoding multiple target polypeptides, wherein the multiple target polypeptides have differences in amino acid sequences from each other and each have, at a terminus, a heterologous peptide of a common sequence wherein the heterologous peptide is shorter than 50 amino acids;
a step of adding a reporter polypeptide, which binds to the heterologous peptide, to a sample of multiple clones derived from the population of thermophile;
a step of detecting the amount of binding of the reporter polypeptide in the sample of multiple clones and identifying one or more clones having an elevated amount of binding to the reporter polypeptide as clones of the thermophile which have accumulated thermostable target polypeptides which are resistant to the screening temperature.

[2] The method of screening according to [1], wherein the thermophile is a genus Geobacillus thermophile.

[3] The method of screening according to [1] or [2], wherein the amount of binding of the reporter polypeptide is detected as the amount of enzymatic activity or fluorescence generated by the reporter polypeptide.

[4] The method of screening according to any one of [1] to [3], wherein the heterologous peptide is a smaller fragment of a split reporter and the reporter polypeptide is a larger fragment of the split reporter.

[5] The method of screening according to [4], wherein the heterologous peptide is an α fragment of a β-galactosidase and the reporter polypeptide is an ω fragment of the β-galactosidase, and wherein the amount of binding is detected as the amount of enzymatic activity generated by a complementation of the α fragment and the ω fragment.

[6] The method of screening according to [4], wherein the heterologous peptide is a GFP11 fragment of a split GFP and the reporter polypeptide is a GFP1-10 fragment of the split GFP, or the heterologous peptide is a fragment of a different GFP-derived split fluorescent protein corresponding to a GFP11 fragment and the reporter polypeptide is a fragment of the different GFP-derived split fluorescent protein corresponding to a GFP1-10 fragment, and
wherein and the amount of binding is detected as the amount of fluorescence generated by self-assembly of the heterologous peptide and the reporter polypeptide.

[7] The method of screening according to any one of [1] to [6], further comprising, prior to the step of culturing, a step of transforming the population of thermophile with the nucleic acid library.

[8] A kit for performing the method of any one of [1] to [7], the kit comprising a plasmid vector and instructions for use,
wherein the plasmid vector comprises a nucleic acid sequence encoding the heterologous peptide and is configured to be capable of expressing, in the thermophile, the target polypeptide fused at the terminus to the heterologous peptide when a nucleic acid encoding the target polypeptide is inserted into the plasmid vector,
wherein the instructions for use comprise instructions for performing each step of the method.

[9] The kit according to [8], further comprising the reporter polypeptide or a nucleic acid encoding the reporter polypeptide.

[10] The kit according to [8] or [9], further comprising one or both of: the thermophile which has not been subjected to the transformation; and an error-prone DNA polymerase.

### BRIEF DESCRIPTIONS OF THE FIGURES

FIG. 1 FIG. 1a shows results of the experiments in which the ω fragment of LacZ and the X-gal substrate were added to the colonies of the thermophile which had been cultured under a high-temperature condition while expressing non-thermostable PyrF_{A} (left) or thermostable PyrFv (right), each fused to the α fragment of LacZ. On the plate on the right, the presence of the accumulated α fragment fusion protein is detected through α complementation with the ω fragment (blue), whereas on the plate on the left, there is poor detection by α complementation due to the lack of accumulation of the α fragment fusion protein. In FIG. 1b, the thermophile was cultured under a high-temperature condition while expressing the Venus protein with (top) or without (bottom) the LacZ α fragment tag, and the subsequently prepared bacterial lysates were serially diluted and spotted as dot blots. The thermostable Venus accumulated in the samples and maintained the fluorescent activity (left). The enzymatic activity provided by α complementation and showing correspondence to the fluorescent activity could also be detected, the enzymatic activity being dependent on the presence of the LacZ α fragment (right).

FIG. 2 shows positions of the mutations in the novel thermostable PyrF protein which was identified and isolated in the example screening. The positions and types of mutations in the amino acid residues relative to the wildtype PyrF are shown in the balloons. Gray balloons indicate synonymous mutations, i.e., mutations which caused changes in the nucleic acid sequences but not in the amino acid residues.

FIG. 3 In FIG. 3a, the non-thermostable PyrF_{A} and the thermostable PyrFv, each fused to the GFP11 fragment of the split GFP, were expressed by the thermophile in liquid cultures under a high-temperature condition, and the GFP1-10 fragment was then added, and the results of measuring GFP fluorescence over time are shown. In the PyrFv sample, self-assembly of the accumulated GFP11 fusion protein and the GFP1-10 reconstituted GFP and thus exhibited relatively high levels of fluorescence. FIG. 3b shows an experiment which corresponded to that of FIG. 3a but performed on solid culture media. A solution of GFP1-10 was added to the plates of solid culture media following a high-temperature culturing and GFP fluorescence was detected by a fluorescence microscope. In the figure, “pHLC” represents a control experiment in which the bacteria were transformed with the plasmid vector pHLC having only the GFP11-coding sequence and lacking a nucleic acid insertion encoding PyrF.

### DETAILED DESCRIPTION OF THE INVENTION

In one aspect, the present disclosure provides a method of screening for a thermostable polypeptide. The method comprises a step of culturing, at a screening temperature of 45 °C or higher, a population of thermophile transformed with a nucleic acid library comprising multiple nucleic acids encoding multiple target polypeptides.

Since a protein is composed of a polypeptide, the terms polypeptide and protein may be used interchangeably in the present disclosure. The term “target” is used to refer to the polypeptides that are subjected to the screening for distinguishing the thermostable ones from those that are not thermostable.

The term nucleic acid library means, as normally understood by a person skilled in the art, a population of nucleic acids containing multiple nucleic acids. What is meant by “multiple nucleic acids” herein is not that there are multiple copies (or multiple molecules) of the same nucleic acid having the same sequence, but that there are nucleic acids having different sequences from each other. The possibility for the presence of multiple copies of the same nucleic acid having the same sequence within the nucleic acid library is also not excluded. The number of multiple nucleic acids comprised in a nucleic acid library can be, for example, at least ten to the power of one, two, three, four, five, or six, or greater. The multiple nucleic acids have different nucleic acid sequences from each other and thus can encode multiple target polypeptides having different amino acid sequences. However, due to codon degeneracy, it is also possible for different nucleic acid sequences to encode a same polypeptide. “Multiple nucleic acids” in the present embodiments may be typically, but not necessarily limited to, those resulting from introduction of random mutations into a coding nucleic acid of the same target polypeptide,

In the present embodiments, the multiple target polypeptides as described above have different amino acid sequences from each other, but are configured to have, at a terminus, a heterologous peptide of a common sequence wherein the heterologous peptide is shorter than 50 amino acids. The terminus can be C-terminus of N-terminus. A “heterologous” peptide herein means a peptide that is not found fused to the target polypeptide in nature. Therefore, a “heterologous peptide” does not necessarily require that it is a peptide originating from a different species than the organism from which the target polypeptide is derived, or a peptide originating from a different species than the thermophile used for the screening. The heterologous peptides in the present embodiments are peptides known to bind to the reporter polypeptides as described below, and various heterologous peptides can be chosen based on knowledge of those skilled in the art. The length of the heterologous peptide is shorter than 50 amino acids, and may be for example 45 amino acids or shorter, 40 amino acids or shorter, or 35 amino acids or shorter. The minimum length of the heterologous peptides required for the binding to the reporter polypeptide should be self-evident to the skilled person. The length of the heterologous peptide is usually 6 amino acids or longer and may be 10 amino acids or longer. The fact that such a short peptide “tag” can be fused to the terminus without impairing the function of the body of the polypeptide is in and of itself widely accepted in the art. Such a short heterologous peptide, in comparison to a protein which is a longer polypeptide, does not involve high order folding in itself, and thus appears to be substantially unaffected by the exposure to the screening temperature.

The heterologous peptide may be for example a common peptide tag available with a corresponding, specific binding antibody. Examples of such peptide tags include, but are not limited to, FLAG tag, HA tag, Myc tag, His tag, and V5 tag. In another example, a biotin acceptor peptide which is recognized/bound and biotinylated by biotin ligase (Biotechnol. 2009, 26:215-221) may be used as the heterologous peptide.

Examples of heterologous peptides in particularly preferable embodiments are, as described below, an α fragment of β-galactosidase (also called LacZ), and a GFP11 fragment of split GFP (green fluorescent protein) as well as corresponding fragments of other GFP-derived split fluorescent proteins. These are the smaller fragments of “split reporters” in the systems generally referred to as protein-fragment complementation assays (PCAs). Many examples of protein-fragment complementation assays are known to those skilled in the art, in which two non-functional polypeptide fragments, resulting from splitting of a full-length protein, are complementarily assembled to regenerate the function (e.g., enzymatic activity or fluorescence) of the full-length protein (Nat Rev Drug Discov. 2007, 6:569-582). The smaller fragments (i.e., of the shorter amino acid sequences) and the larger fragments (i.e., of the longer amino acid sequences) of these split reporters in the respective combinations may be used as the heterologous peptides and the reporter polypeptides in the present embodiments, respectively. Thus, in the present disclosure, the term “split reporter” refers to a combination of complementary polypeptide fragments which are known to be usable in a protein-fragment complementation assay. For example, the smaller fragment of the split luciferase (ACS Chem. Biol. 2016, 11, 400-408; ACS Chem. Biol. 2018, 13, 467-474) may be used as the heterologous peptide in the present embodiments. These heterologous peptides are especially preferable because they can generate detectable activity or signal by binding to the reporter polypeptides that do not have detectable activity or signal. In other words, they can generate detectable activity or signal only when the heterologous peptide and the reporter polypeptide are properly bound, and they can therefore advantageously reduce false positive noises in the screening significantly.

A given population of microbe, such as thermophile, can be transformed with a nucleic acid library comprising multiple nucleic acids by a method known to a person skilled in the art. In the present disclosure, transformation means introducing polypeptide-coding nucleic acids to the microbe from outside to induce a condition in which the polypeptide can be expressed in the microbe. Many methods of transformation are known, and for example transformation by electroporation, or transformation via conjugation from another type of bacterium (e.g., E. coli), may be used. It is appreciated that the multiple nucleic acids are typically introduced in the form of plasmids into the thermophile for effecting transformation. In the plasmid, the target polypeptide and the heterologous peptide are encoded such that they are fused in-frame, and this coding sequence is placed under the control of a suitable promoter known to the skilled person. As a result of the transformation, the different nucleic acids of the nucleic acid library are incorporated into different individuals of the thermophile separately. Thus, it is appreciated that the different individuals of the thermophile can now express the target polypeptides having respectively different amino acid sequences (as the fusions to the common heterologous peptide mentioned above).

In one embodiment, the method further comprises, prior to the step of culturing at the screening temperature, a step of transforming the population of thermophile with the nucleic acid library. The transformation may be performed all at once, but alternatively, one portion and another portion of the population of thermophile may be separately transformed with one portion and another portion of the nucleic acid library, respectively. The method may further comprise, prior to the step of transforming, a step of preparing the nucleic acid library by incorporating the multiple nucleic acids (such as nucleic acids resulting from random mutagenesis in the nucleic acid encoding the target polypeptide) into transformation plasmids. Further prior to this step, the method may comprise a step of introducing random mutations to the nucleic acids encoding the target polypeptide. Many methods for incorporating nucleic acids into transformation plasmids and for introducing random mutations to nucleic acids are known to those skilled in the art and can be used for the purpose of the present embodiments. For example, random mutations can be introduced to nucleic acids by an error-prone PCR method using an error-prone DNA polymerase.

The thermophiles are microbes having optimal growth temperatures of no lower than 45 °C, or no lower than 50 °C, and may be for example thermophilic bacteria such as bacteria of the genus Geobacillus. Examples of suitable thermophiles of the genus Geobacillus include, but are not limited to, G. kaustophilus, G. thermodenitrificans, G. subterraneus, G. thermoglucosidasius, G. thermoleovorans, and G. stearothermophilus. Other examples of the thermophiles that may be used in the present embodiments include Thermus thermophilus, Clostridium thermocellum, Thermoanaerobacter sp. X514, Thermoanaerobacterium saccharolyticum, Thermotoga maritima, Thermotoga neapolitana, Pyrococcus abyssi, Pyrococcus furiosus, Sulfolobus acidocaldarius, Sulfolobus solfataricus, and Thermococcus kodakarensis.

The temperature and the time of screening may be determined by a person skilled in the art as appropriate depending on such factors as specific microbial species used, types of the target proteins, and temperatures aimed at for the thermostability. The screening temperature may be for example 47 °C or higher, 48 °C or higher, 50 °C or higher, 55 °C or higher, 60 °C or higher, or 65 °C or higher. It is common technical knowledge that even thermophiles have upper limits for the temperature at which each strain can tolerate, and therefore the upper limits of the screening temperatures should be self-evident to those skilled in the art. The screening temperature is usually no higher than 80 °C, more typically no higher than 74 °C. The time length of the culturing performed at the screening temperature may be, for example and without limitation, 8 to 48 hours, 10 to 36 hours, or 12 to 24 hours. To culture a population of the thermophile at the screening temperature, it is preferable to start culturing the whole population at the same time, but an alternative embodiment is also possible in which culturing of one portion of the population and culturing of another portion of the population are commenced at different times.

The screening method according to the present embodiments comprises a step of adding a reporter polypeptide, which binds to the heterologous peptide, to a sample of multiple clones derived from the population of thermophile which is from the step of culturing at the screening temperature. In the method of the present embodiments, the reporter polypeptide may be a polypeptide prepared by a conventional technology (for example recombinant expression technology), and it is noteworthy that the reporter polypeptide does not need to be exposed to a high temperature such as the screening temperature. The step of adding the reporter polypeptide may be performed at a temperature lower than the screening temperature, for example between room temperature and 37 °C. If a reporter polypeptide having thermostability is used, it is also possible to perform the step of adding the reporter polypeptide at a temperature similar to the screening temperature.

A person skilled in the art can select, using existing knowledge, various heterologous peptide—reporter polypeptide combinations capable of specific binding. The reporter polypeptide may be for example an antibody (including various antibody fragments known by those skilled in the art to retain the specific binding ability) or the like which specifically binds to the heterologous peptide. For example, if the heterologous peptide is a FLAG tag, an anti-FLAG antibody may be used as a reporter polypeptide. It is also within ordinary skill in the art to produce an antibody which specifically binds to a given peptide tag. In a preferable embodiment, the reporter polypeptide is not an antibody. For example, if the heterologous peptide is a smaller fragment of a split reporter, a larger fragment of the split reporter may be used as a reporter polypeptide. For example, if the heterologous peptide is an α fragment of a β-galactosidase, an ω fragment of the β-galactosidase may be used as a reporter polypeptide. If the heterologous peptide is a GFP11 fragment of a split GFP (or a corresponding fragment of another GFP-derived split fluorescent protein), a GFP1-10 fragment of the split GFP (or a corresponding fragment of another GFP-derived split fluorescent protein) may be used as a reporter polypeptide. If the heterologous peptide is a smaller fragment of a split luciferase, a larger fragment of the split luciferase may be used as a reporter polypeptide. If a biotin acceptor peptide is used as a heterologous peptide, a biotin ligase (BirA) may play a role of a reporter polypeptide. The binding of the biotin ligase is believed to be transient, but the amount of binding of the reporter polypeptide may be represented by the amount of biotinylation.

A clone means a group which has resulted from multiplication of one progenitor (for example one nucleic acid molecule or one cell) and consequently has an identical genotype within, and a clone can be present for example as a population of nucleic acid molecules or a colony of cells. The sample of clones derived from the population of thermophile may be, for example, individual colonies formed on a sold culture medium or suspensions of the individual colonies. In an embodiment, the cells in the sample are lysed. In another embodiment, the cells are permeabilized, for example by addition of a surfactant. It has been found that, with simple adjustment of the surfactant concentration, it is possible to permeabilize part or all of the cells while part or all of the cells in the clone remain viable. Such an embodiment is advantageous because it allows direct recovery of viable cells from the clone (e.g., a colony on a culture plate) determined to be positive for thermostability by the reporter polypeptide and therefore significantly improves the efficiency of the screening. Alternatively, it is also possible to make two or more replicas of the colonies or suspensions of viable cells and test thermostability on only some of the replicas. It is appreciated by a person skilled in the art that the cell suspension or lysate can be further adsorbed on a solid substrate such as PVDF and nitrocellulose.

In a screening for thermostable polypeptides, typically, the vast majority of the multiple target polypeptides may be non-thermostable. However, if there are a small number of thermostable target polypeptides among them, the thermostable ones may accumulate with the samples of the host thermophile clones, bind to the reporter polypeptides added in the above step, and thereby make themselves detected (the term accumulate in this case may be also applied to a polypeptide which is secreted extracellularly). In contrast, when non-thermostable heterologous polypeptides are forced to be expressed in a thermophile, the conventional thinking has been that they accumulate while causing aggregation or the like in the denatured or misfolded forms. However, in the course of researches attempting to express various heterologous polypeptides in thermophiles, the present inventor observed that, as a rule, polypeptides that misfolded under the high temperatures apparently did not accumulate but instead were subjected to clearance relatively quickly thereby failing to be even detected. For example, when a protein was forced to be expressed in a thermophile at different culturing temperatures, the protein failed to be detected in either the soluble fraction or the insoluble fraction beyond certain temperatures. Therefore, it is possible to detect the amount of binding of the reporter polypeptide in the sample of multiple clones to identify one or more clones, which show relatively elevated amount of binding to the reporter polypeptide, as thermophilic clones which were expressing thermostable versions of the target polypeptides resistant to the screening temperature, i.e., which has accumulated the thermostable target polypeptides. Detecting the amount of binding may include not only distinguishing the clones showing binding in comparison to the clones not showing significant binding, but also quantifying the amount of binding.

A person skilled in the art can select a suitable method for detecting the amount of binding of the reporter polypeptide based on ordinary knowledge. For example, if the reporter polypeptide is an antibody, the antibody itself or a secondary antibody which binds to it can be used in the form conjugated to a detectable label or enzyme. In some embodiments, the amount of binding of the reporter polypeptide may be detected as the amount of enzymatic activity or fluorescence generated by the reporter polypeptide. In this context, the amount of enzymatic activity or fluorescence generated by the reporter polypeptide refers to the amount of enzymatic activity or fluorescence which became present and concentrated at the site of the sample of interest due to the presence of the reporter polypeptide concentrated at the site. An exemplary case in which a reporter polypeptide generates enzymatic activity or fluorescence is where the reporter polypeptide is an antibody and said antibody or a secondary antibody that binds to it is conjugated to an enzyme or a fluorescent dye.

Depending on the types of the reporter and the label, adding the reporter polypeptide and detecting the amount of its binding may be performed on the colonies formed on solid culture media, or in the suspensions or lysates of the colonies, or on solid substrates (e.g., PVDF or nitrocellulose membranes) to which any of the above have been adsorbed. Adsorbing the sample to the solid substrate may be useful for improving sensitivity of the detection of positive clones by washing away the reporter polypeptides that did not bind following the addition. It is appreciated by a person skilled in the art that various known high-throughput systems capable of simultaneous analysis of many samples may be compatible with implementation of the screening method of the present embodiments.

The reporter polypeptide is usually added externally to the sample of multiple clones derived from the population of thermophile following the culturing at the screening temperature. However, in the case where the reporter polypeptide itself is resistant to the screening temperature, for example in an embodiment which uses a LacZ ω fragment derived from a thermophile (e.g. Geobacillus stearothermophilus) as a reporter polypeptide, the reporter polypeptide may be expressed by the thermophile itself during the culturing at the screening temperature to accomplish the addition of the reporter polypeptide. Thus, in certain embodiments, the sample of multiple clones derived from the population of thermophile may be the population of thermophile itself under culture, wherein the reporter polypeptide may be expressed by the transformed thermophile itself, and the step of culturing and the step of adding the reporter polypeptide may take place simultaneously.

Clones having an elevated amount of binding to the reporter polypeptide may refer to the clones in which the binding is occurring in distinction from the clones in which the binding is not occurring, or the clones showing greater detection levels of binding than other clones when there are multiple clones showing detectable binding (e.g., when there is a background level of binding). The clones having an elevated amount of detectable binding to the reporter polypeptide may be called “positive” clones in the context of the screening. Typically, a vast majority of the population of the thermophile screened is negative, and only a limited minority is found to be positive. For example, on a plate of solid culture medium having a diameter of about 10 cm and having many densely formed colonies, only several to a few dozen positive clones may be found.

Once the clones of the thermophile which have accumulated thermostable versions of target polypeptides are identified, the skilled person can easily determine the sequences of the target polypeptides corresponding to those clones. For example, the nucleic acid originating from the nucleic acid library and present in the sample of the clone can be amplified by PCR with specific primers, allowing determination of the sequence of the thermostable target polypeptide. Amplification and sequencing can be also carried out after the plasmid originating from the nucleic acid library and present in the sample of the clone is recovered and used for new transformation. Alternatively, if the cells have not been lysed or if a replica of the sample has been prepared, there are viable microbes in the sample, and therefore propagating the viable microbes would be useful for the analysis including sequence determination.

In a particularly preferable embodiment of the invention, the heterologous peptide is an α fragment of a β-galactosidase, the reporter polypeptide is an ω fragment of the β-galactosidase, and the amount of binding is detected as the amount of enzymatic activity produced by α-complementation of the α fragment and the ω fragment. α-complementation of β-galactosidase is a classic, well-known phenomenon (J Mol Biol (1967) 24(2):339-43; J Biol Chem (1981) 256(13):6804-6810), and also a classic example of split reporter, and a person skilled in the art can clearly recognize an α fragment and an ω fragment of a β-galactosidase. An original full-length β-galactosidase enzyme has a length of about 1000 amino acids. The ω fragment is a large fragment corresponding to the majority of the full-length 3-galactosidase enzyme, but lacks the enzymatic activity because it lacks the small α fragment portion. The α fragment, having a length of less than 50 amino acids, also does not have the enzymatic activity, of course. However, when the α fragment and the ω fragment assemble, an enzymatic activity similar to that of the full-length β-galactosidase enzyme can be restored.

Use of α-complementation can significantly increase sensitivity of the detection of the positive clones, as a detectable β-galactosidase is produced only in the clones in which the thermostable polypeptides have accumulated to be able to bind to the reporter polypeptide. For example, a filter paper may be placed on the solid culture medium plate having the colonies of the thermophile which have been cultured at the screening temperature, then a surfactant (e.g., a 0.01 to 0.5% (w/v) SDS aqueous solution) for permeabilizing the cells is applied to the filter paper, followed by direct application of a solution of the ω fragment thereto. Further, the application thereto of X-gal which is a chromogenic substrate for the β-galactosidase will allow the detection of a few positive colonies stained in blue color amidst the large majority of the colonies in white color. The applications of these solutions may be achieved by, e.g., spraying using a sprayer.

The β-galactosidase from which the α fragment and the ω fragment of the present embodiments are derived may be a bacterial β-galactosidase such as, but not necessarily limited to, a β-galactosidase from a thermophile, which may be a β-galactosidase from a bacterium of the genus Geobacillus. In an embodiment, the α fragment has SEQ ID NO: 1 or a sequence having sequence identity of 80% or higher, 90% or higher, 95% or higher, or 99% or higher to SEQ ID NO: 1, and the ω fragment has SEQ ID NO: 2 or a sequence having sequence identity of 80% or higher, 90% or higher, 95% or higher, or 99% or higher to SEQ ID NO: 2. It has been found that the α fragments, together with the ω fragments, can cause α-complementation either fused to the N-terminal ends or C-terminal ends of the target polypeptides, but fusing the α fragment to the C-terminal end of the target polypeptide may be more preferable. It is appreciated by a person skilled in the art that an N-terminal-most methionine, a tag for purification or for detection, a linker, etc. may be added to these sequences as needed.

In another preferable embodiment, the heterologous peptide is a GFP11 fragment of a split GFP, the reporter polypeptide is a GFP1-10 fragment of the split GFP, and the amount of binding is detected as the amount of fluorescence produced by self-assembly of the GFP11 fragment and the GFP1-10 fragment. The concept of split GFP and the fragments involved in it have been described by Cabantous et al. and known to those skilled in the art (Nat Biotechnol (2005) 23(1):102-107). A natural GFP has a length of 238 amino acids. The GFP1-10 fragment is a large fragment corresponding to the majority of the full-length GFP, but lacks the fluorescent activity because it lacks the small GFP11 fragment portion. The small GFP11 fragment, having a length of less than 50 amino acids, also does not have the fluorescent activity, of course. However, when the GFP1-10 fragment and the GFP11 fragment assemble, a fluorescent activity similar to that of the full-length GFP can be restored. In an embodiment, the GFP11 fragment has SEQ ID NO: 3 or a sequence having sequence identity of 80% or higher, 90% or higher, 95% or higher, or 99% or higher to SEQ ID NO: 3, and the GFP1-10 fragment has SEQ ID NO: 4 or a sequence having sequence identity of 80% or higher, 90% or higher, 95% or higher, or 99% or higher to SEQ ID NO: 4. The GFP11 fragment is preferably fused to the C-terminal end of the target polypeptide.

Many GFP-derived fluorescent proteins are known (e.g., CFP, YFP, and the like) which have been created by introducing point mutations to the GFP to alter the wavelength (color) of the fluorescence. These different GFP-derived fluorescent proteins have substantially the same overall three-dimensional structures compared to GFP, and the skilled person would therefore appreciate that the split fluorescent protein corresponding to the split GFP can be provided. Thus, in an embodiment, the heterologous peptide is a fragment of a different GFP-derived split fluorescent protein corresponding to the GFP11 fragment, the reporter polypeptide is a fragment of the different GFP-derived split fluorescent protein corresponding to the GFP1-10 fragment, and the amount of binding is detected as the amount of fluorescence produced by self-assembly of the heterologous peptide and the reporter polypeptide. Here, “a different GFP-derived split fluorescent protein” means a fluorescent protein which is a derivative of the GFP with an altered sequence (and typically an altered fluorescence wavelength) due to introduction of a mutation compared to the GFP and which is split as in the split GFP.

In another aspect of the present disclosure, a kit for performing the method embodiments described above is provided. The kit may comprise at least a plasmid vector and instructions for use. The plasmid vector is characterized by comprising a nucleic acid sequence encoding the heterologous peptide described above, and being configured to express in a thermophile a target polypeptide fused to said heterologous peptide at its terminus, when a nucleic acid encoding the target polypeptide is inserted in the plasmid vector. Other common elements such a plasmid expression vector may comprise (a replication origin, a promoter, a drug resistant gene for selection, etc.) are well-known to those skilled in the art.

The instructions for use include instructions to perform the steps of the method described above. It should be appreciated that such instructions may comprise, for example, descriptions for one or more or all of: preparing the nucleic acid library by incorporating the multiple nucleic acids (such as nucleic acids resulting from random mutagenesis in the nucleic acid encoding the target polypeptide) into the plasmid vector; transforming the population of thermophile with the nucleic acid library; culturing at the screening temperature the population of the transformed thermophile; preparing the sample of multiple clones from the population of the thermophile following the culturing; adding the reporter polypeptide to the sample of multiple clones derived from the population of the thermophile; detecting the amount of binding of the reporter polypeptide in the sample of multiple clones; and identifying one or more clones having an elevated amount of binding to the reporter polypeptide as clones of the thermophile which have accumulated thermostable target polypeptides which are resistant to the screening temperature. The instructions for use may further include a description for introducing random mutations to the nucleic acid encoding the target polypeptide.

The kit of the present embodiment may further comprise the reporter polypeptide or a coding nucleic acid thereof (i.e., a nucleic acid from which a person skilled in the art can prepare the reporter polypeptide). The kit of the present embodiment may comprise cells of the thermophile which are before the transformation. The kit of the present embodiment may comprise an error-prone DNA polymerase for introducing random mutations to the nucleic acid encoding the target polypeptide. These and other components that may be included in the kit according to particular embodiments will be apparent to the skilled person in view of the descriptions of the method embodiments provided above.

Below, examples are provided to further illustrate embodiments of the invention of the present disclosure in greater detail, but these examples are provided merely as representative experimental examples and the invention is not limited to these specific examples.

### EXAMPLES

In this example, a proof-of-concept of the screening method of the disclosure is provided, using the PyrF protein originating from Bacillus subtilis (a non-thermophilic hay bacillus) and described by Non-Patent Document 2, of which the present inventor is the main author. The amino acid sequence of the PyrF wildtype protein and the coding nucleic acid sequence thereof are provided as SEQ ID NOS: 5 and 6, respectively. These sequences can be referenced in the National Center for Biotechnology Information, U.S.A., database, under the genome sequence locus tag number BSU_15550 and under NP_389438 and NC_000964 (REGION: 1628622 . . . 1629341). While the wildtype PyrF_{A} is not thermostable, Non-Patent Document 2 identified/isolated a thermostable variant of PyrF_{A}, called PyrFv, by combining the use of an error-prone strain of the thermophile Geobacillus kaustophilus, characterized by the increased mutation rate, with the methodology designed to select individuals that can survive under the selective pressure depending on the PyrF enzymatic activity. In the subsequent researches, the inventor has found a very simple but previously not consciously appreciated prevalent rule that, when heterologous proteins are expressed in a thermophile, thermostable proteins would accumulate in the culture but non-thermostable proteins would not just turn into denatured or aberrantly folded forms but such forms seem to be subject to clearance and fail to even accumulate. This prevalent rule is observed for PyrF_{A} and PyrFv as well.

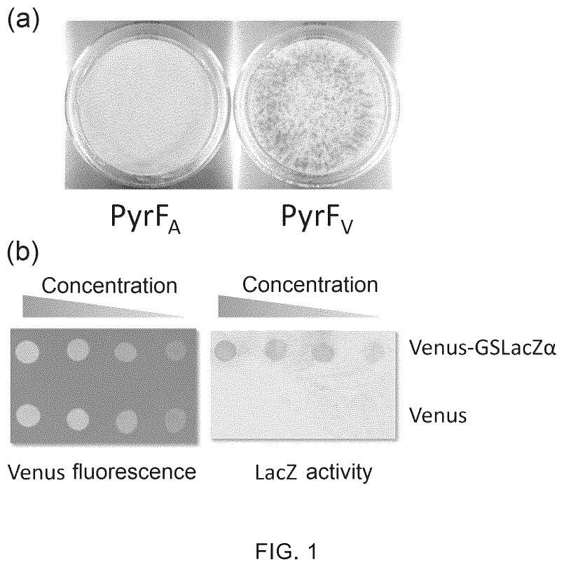

In a preliminary experiment represented by FIG. 1a, the coding genes for PyrF_{A} and PyrFv were inserted and integrated to the plasmid vectors for expression in the thermophiles. These plasmid vectors encode the α fragment of β-galactosidase (lac Z) from Geobacillus stearothermophilus (SEQ ID NO: 1; 37 amino acids). Thus, the target polypeptide encoded by the inserted gene is configured to be expressed with the α fragment fused at the C-terminus. Geobacillus thermodenitrificans K1041 strain was transformed with this plasmid vector and cultured on a 90 mm plate of solid culture medium or in a liquid culture medium at 50 °C. A filter paper was placed on the surface of the solid culture medium which had formed the colonies, and 700 μL of an SDS aqueous solution (0.1% (w/v)) was sprayed onto the filter paper, and incubation at the temperature was continued (5 min). Next, an aqueous solution (700 μL) of the ω fragment (SEQ ID NO: 2, bearing a His tag at the N-terminus) was sprayed, followed by incubation at the temperature (10 min). Finally, a DMSO solution of X-gal (700 μL; 5.0 mg/mL) was sprayed, and incubation at the temperature was continued until development of blue color became apparent on the filter paper over the colonies. As shown in FIG. 1a, in the colonies which had expressed and accumulated the thermostable PyrFv on the solid culture medium, the ω fragment was bound at a high level to cause α complementation, resulting in the strong detection of the specific β-galactosidase activity (right, blue). In contrast, in the colonies expressing the non-thermostable PyrF_{A}, hardly any β-galactosidase activity is detected despite the addition of the ω fragment. High sensitivity of this screening, or its low false-positive level, is quite striking. A low copy number plasmid and a high copy number plasmid were both tested but gave essentially the same results. Further, an experiment was performed in which the liquid culture was combined with a surfactant (SDS at 0.01% final concentration), an aqueous solution of the ω fragment, and an aqueous solution of the β-galactosidase substrate o-nitrophenyl-β-D-galactopyranoside, to measure color development of the substrate. In this experiment, too, a high β-galactosidase activity specific to PyrFv was detected (data not shown).

Next, an error-prone PCR was performed to introduce random mutations to the nucleic acid encoding the non-thermostable PyrF_{A} as a template. The population of the nucleic acids following the mutagenesis was inserted into the plasmid vector encoding the α fragment (corresponding to the heterologous peptide described in the present specification) to prepare a nucleic acid library. This nucleic acid library was propagated via transformation of the E. coli DH5α strain, followed by transformation of the E. coli HST04 strain (to remove DNA methylation generated in the DH5α strain), and finally transformation of the thermophile Geobacillus thermodenitrificans K1041 strain was carried out by electroporation. Following the culturing at the temperature of 50 °C (screening temperature), the ω fragment (reporter polypeptide)—X-gal assay on solid culture medium plates, as described above, was used to perform primary screening of 1×10⁴ clones, which identified 9 colonies as candidate colonies which appeared blue-positive, i.e., which appeared to have accumulated mutant PyrF fusion proteins resistant to the screening temperature. These candidate colonies were collected by picking and cultured in liquid media, re-plated on solid culture media to form colonies again, and replicas of these plates were produced. Each candidate strain was then subjected to secondary screening on one plate to narrow down the positive candidates to 8 strains. Plasmid DNAs were extracted from the colonies on the other replicas for sequence determination.

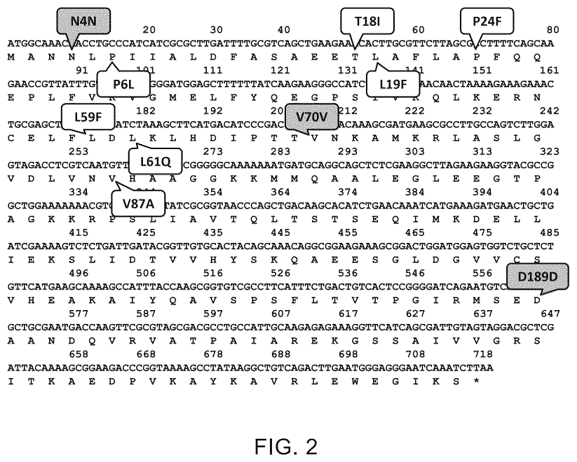

As a result, sequence changes were found in the coding nucleic acid of PyrF_{A} in 7 candidate strains out of 8. In fact, these 7 strains all had identical mutations in the sequence, and were therefore presumed to represent separate colonies propagated from the same clone. The mutant PyrF which was common to these 7 strains is called PyrF_{X}. Positions of the amino acid changes in PyrF_{X} relative to the wildtype PyrF_{A} are shown in FIG. 2. The positions of the amino acid changes in PyrF_{X} did not coincide with those found in PyrFv.

PyrF_{A}, PyrFv, and the newly isolated/identified PyrF_{X}, each in a His-tagged form, were recombinantly expressed in E. coli or in a thermophile (G. thermodenitrificans) using conventional methods, and PyrFs were purified from soluble fractions. However, PyrF_{X} did not emerge in the soluble fraction in E. coli and therefore purification was prevented in that case. Table 1 below shows the results of: the assay of measuring the midpoint of denaturing temperatures based on the detection of exposure of hydrophobic regions in denatured proteins by the binding of a hydrophobic fluorescent dye (left; Protein Thermal Shift™ Dye Kit (Thermo Fisher Scientific, 4461146); and the assay of determining the half-precipitation temperature which also provides an index of denaturing point (right; the sample was heat-treated for 1 hour at different temperatures on Thermal Cycler Dice Gradient (Takara Bio), followed by centrifugation, and the supernatant, i.e. non-precipitated protein, was quantified by SDS-PAGE). As clearly seen in Table 1, PyrF_{X} had attained the level of thermostability equal to or greater than that of PyrFv.

TABLE 1

| | | | | | Half |
|---|---|---|---|---|---|
| | Host for | Denaturing | | Host for | precipitation |
| Enzyme | production | temperature | Enzyme | production | temperature |
| PyrF_{X} | E. coli | ND | PyrF_{X} | E. coli | ND |
| PyrF_{A} | E. coli | 55 ± 0 °C | PyrF_{A} | E. coli | 28 ± 0 °C |
| PyrF_{V} | E. coli | 63 ± 0 °C | PyrF_{V} | E. coli | 41 ± 2 °C |
| PyrF_{X} | Thermo- | 63 ± 0 °C | PyrF_{X} | Thermo- | 41 ± 0 °C |
| | phile | | | phile | |
| PyrF_{A} | Thermo- | 55 ± 0 °C | PyrF_{A} | Thermo- | 26 ± 0 °C |
| | phile | | | phile | |
| PyrF_{V} | Thermo- | 63 ± 0 °C | PyrF_{V} | Thermo- | 38 ± 0 °C |
| | phile | | | phile | |

Of course, the ability to detect thermostable proteins by means of β-galactosidase (lacZ) α fragment tag is not limited to PyrF. As an example to illustrate this point, the experiment with Venus is described here. Venus is a known yellow fluorescent protein. While most of heterologous proteins not originating from thermophiles do not accumulate when expressed in a thermophile cultured under a high temperature, it has been found that Venus exceptionally has thermostability and can be accumulated in a thermophile cultured under a high temperature. In the experiment of FIG. 1b, Venus fused to the lacZ α fragment was expressed in G. thermodenitrificans cultured at 55 °C, and the cells were then lysed by ultrasonic treatment and the supernatants were collected. This lysate was serially diluted and applied dropwise onto a PVDF membrane to provide a dot blot. As clearly seen in the figure, the α fragment-tagged Venus protein produced under the temperature of 55 °C itself retained the fluorescent activity (FIG. 1b, left), and when the ω fragment was applied to it for binding, the β-galactosidase was reconstituted, resulting in the detection of the enzymatic activity showing correspondence to the fluorescent activity and depending on the presence of the lacZ α fragment (FIG. 1b, right).

In the exemplary experiments described above, the α fragment of β-galactosidase originating from G. stearothermophilus, a thermophile, was used as a heterologous peptide tag to be exposed to the high heat of the screening temperature together with the target polypeptide. However, the heterologous peptide tag is not necessarily limited to this. In the exemplary experiment described next, instead of the α fragment, the GFP11 fragment of split GFP described by Cabantous et al. (SEQ ID NO: 3, 16 amino acid long; 30 amino acid long including the GS-rich linker (SEQ ID NO: 7)) was fused to the C-terminus of PyrF_{A} or PyrFv, and this was expressed in the transformed thermophile G. thermodenitrificans K1041 strain while the strain was cultured in a liquid culture medium at 50 °C for 24 hours. Subsequently, the culture liquid was diluted, supplemented with the SDS surfactant at a final concentration of 0.01-0.02%, and incubated at 37 °C for 5 minutes. A solution of GFP1-10 fragment (SEQ ID NO: 4; with an attached His tag for purification), which had been separately and recombinantly expressed in E. coli and purified, was added to the culture liquid, and the mixture was placed in a 96-well plate with all surfaces painted black (Greiner FLUOTRAC 200). Fluorescence was measured on a Nivo™ multimode plate reader (PerkinElmer). Conditions for the measurement were as follows:
Measurement intervals; 5 min
Excitation light filter: 480/30 nm
Fluorescence filter: 530/30 nm
Dichroic mirror: D500
Temperature: 30 °C
Measurement time (exposure): 200 ms

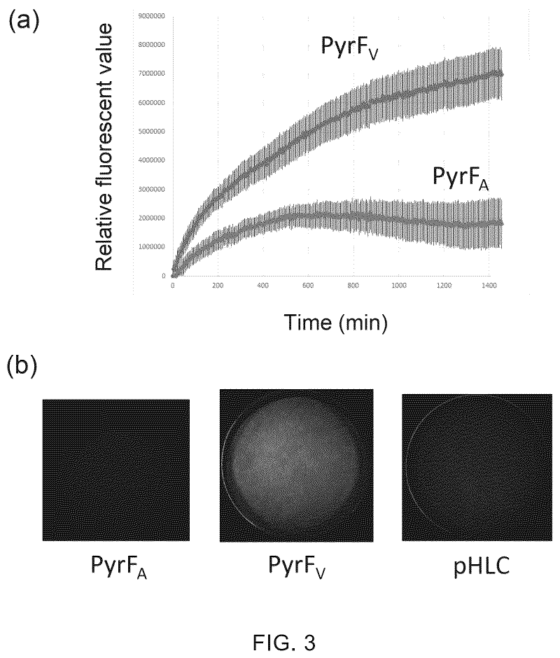

The results of the GFP fluorescence measurements in the liquid culture are shown in FIG. 3a. It can be appreciated that the thermostable PyrFv was accumulated in the culture during the culturing at the high temperature, allowing the binding of GFP1-10 via the GFP11 tag, and therefore relatively strong fluorescence was detected due to the self-assembly-based reconstitution of GFP. In contrast, the non-thermostable PyrF_{A} appeared to have accumulated poorly, as shown by the low fluorescence level.

In the experiment of FIG. 3b, the same transformed strains as above were cultured on solid culture media at 50 °C for 24 hours to allow formation of colonies. Subsequently, a 0.1% SDS aqueous solution was sprayed onto the colonies which were then incubated at room temperature for 5 minutes, and then a solution of the GFP1-10 fragment (0.867 mg/mL) was sprayed followed by further incubation at room temperature for 24 hours. The culture plates were then placed in ImageQuant™ LAS 4000 mini (GE HealthCare) to perform epifluorescence detection (Cy2 mode: Epi-Blue light (460 nm) & Y515 filter; exposure: 0.5 seconds). From FIG. 3b, it can be seen that the results of the fluorescence detection corresponded to those obtained in the liquid cultures. Other detection systems than those demonstrated in these examples can also be employed to detect the amount of binding of the reporter polypeptides in the screening methods.

1. A method of screening for a thermostable polypeptide, the method comprising:
a step of culturing, at a screening temperature of 45 °C or higher, a population of thermophile transformed with a nucleic acid library comprising multiple nucleic acids encoding multiple target polypeptides, wherein the multiple target polypeptides have differences in amino acid sequences from each other and each have, at a terminus, a heterologous peptide of a common sequence wherein the heterologous peptide is shorter than 50 amino acids;
a step of adding a reporter polypeptide, which binds to the heterologous peptide, to a sample of multiple clones derived from the population of thermophile;
a step of detecting the amount of binding of the reporter polypeptide in the sample of multiple clones and identifying one or more clones having an elevated amount of binding to the reporter polypeptide as clones of the thermophile which have accumulated thermostable target polypeptides which are resistant to the screening temperature.

2. The method of screening according to claim 1, wherein the thermophile is a genus Geobacillus thermophile.

3. The method of screening according to claim 1, wherein the amount of binding of the reporter polypeptide is detected as the amount of enzymatic activity or fluorescence generated by the reporter polypeptide.

4. The method of screening according to claim 3, wherein the heterologous peptide is a smaller fragment of a split reporter and the reporter polypeptide is a larger fragment of the split reporter.

5. The method of screening according to claim 4, wherein the heterologous peptide is an α fragment of a β-galactosidase and the reporter polypeptide is an ω fragment of the β-galactosidase, and wherein the amount of binding is detected as the amount of enzymatic activity generated by α complementation of the α fragment and the ω fragment.

6. The method of screening according to claim 4, wherein the heterologous peptide is a GFP11 fragment of a split GFP and the reporter polypeptide is a GFP1-10 fragment of the split GFP, or the heterologous peptide is a fragment of a different GFP-derived split fluorescent protein corresponding to a GFP11 fragment and the reporter polypeptide is a fragment of the different GFP-derived split fluorescent protein corresponding to a GFP1-10 fragment, and
wherein and the amount of binding is detected as the amount of fluorescence generated by self-assembly of the heterologous peptide and the reporter polypeptide.

7. The method of screening according to claim 1, further comprising, prior to the step of culturing, a step of transforming the population of thermophile with the nucleic acid library.

8. A kit for performing the method of claim 1, the kit comprising a plasmid vector and instructions for use,
wherein the plasmid vector comprises a nucleic acid sequence encoding the heterologous peptide and is configured to be capable of expressing, in the thermophile, the target polypeptide fused at the terminus to the heterologous peptide when a nucleic acid encoding the target polypeptide is inserted into the plasmid vector,
wherein the instructions for use comprise instructions for performing each step of the method.

9. The kit according to claim 8, further comprising the reporter polypeptide or a nucleic acid encoding the reporter polypeptide.

10. The kit according to claim 8, further comprising one or both of: the thermophile which has not been subjected to the transformation; and an error-prone DNA polymerase.


SEQUENCE LISTING (7 sequences)

SEQ ID NO: 1 - protein, 37 aa, synthetic construct
KKTLTTPTLEWLSDPRVFGVNRLPAHSDHIYYETLEE

SEQ ID NO: 2 - protein, 975 aa, synthetic construct
AKSGVSMSMRYELNGNWKFHYAVNPASRPAKFYQLDFDCSNWGDISVPGHIQLQGYGTPQYVNTMYPWDGHCDVRPPEVPMDYNPVGSYIKIFNLPERMKGKPVYISFQGVESAFYVWLNGQFVGYSEDSFTPAEFELTPFIKDGENKLAVEVYQYSTGSWLEDQDFWRFSGIFRDVYLYTVPDIHVYDLHVRAELDSSYTRGMLTADLTFLSPVPAGSRFIAELYDAKGDLVAKKDGELHGEKGTFSIAVDEPELWSAEKPYLYKLYIQIFNEFGQLVEVVPQKVGFRRFEIINKVMHLNGKRIVFKGVNRHEFHCRRGRAITKEDMLWDIKTLKRHNINAVRTSHYPNQSYWYELCDEYGIYVIDEMNLETHGTWQKMGRVDPTWNVPGNNREWQDIVMDRAISMFERDKNHPSILIWSCGNESYAGEVLLNVANYFRSVDPSRLVHYEGVFHAREYNDISDIESRMYAKPHEIEKYLTENPDKPYISCEYMHAMGNSLGGMYKYTDLEQKYPMYQGGFIWDFIDQALVKKDRYGKEFLAYGGDFGDRPTDYGFCTNGIVYANRELSPKMQEVKYLYQNFKLSPDQYGVNIRNESLFTDTSEYDLEYVLFLEGRELYRSKLNVSVAPQEEKRVEFDWPLGMMADAGEYCIHASFKLKEKTLWADAGYEVAFGQFVFQIGSKEFVVPSGKLKVVHGDVNIGVHGKDFTIMFSKQAGSLVSMNYAGKEMIAVPPAPLFWRATTDNDRGFLQDFYSGLWYAASLARKCVKVDVKEEAAQVNVTFTYKFSINPELEVKIGYTVYADGSIRVKAVYHGVENLPSMPIFAVSFKVPADYNQLEWYAMGPEENYVDRSKGARLGIFRNQVSDNVSRYLKPQESGNRTGVRKVSLTNNNGQGIKISSITEPLECNFSPYTAFELEHANHHYELPNIHYTVVTVAGRQMGVGGDDSWGAPVHDEYVIKADQDMEFEFMIHKV

SEQ ID NO: 3 - protein, 16 aa, synthetic construct
RDHMVLHEYVNAAGIT

SEQ ID NO: 4 - protein, 214 aa, synthetic construct
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEK

SEQ ID NO: 5 - protein, 239 aa, Bacillus subtilis
MKNNLPIIALDFASAEETLAFLAPFQQEPLFVKVGMELFYQEGPSIVKQLKERNCELFLDLKLHDIPTTVNKAMKRLASLGVDLVNVHAAGGKKMMQAALEGLEEGTPAGKKRPSLIAVTQLTSTSEQIMKDELLIEKSLIDTVVHYSKQAEESGLDGVVCSVHEAKAIYQAVSPSFLTVTPGIRMSEDAANDQVRVATPAIAREKGSSAIVVGRSITKAEDPVKAYKAVRLEWEGIKS

SEQ ID NO: 6 - DNA, 720 bp, Bacillus subtilis
  source 1..720: mol_type = genomic DNA
atgaaaaacaacctgcccatcatcgcgcttgattttgcgtcagctgaagaaacacttgcgttcttagcgccttttcagcaagaaccgttatttgtaaaggttgggatggagcttttttatcaagaagggccatctatcgtgaaacaactaaaagaaagaaactgcgagctatttttagatctaaagcttcatgacatcccgactactgtaaacaaagcgatgaagcgccttgccagtcttggagtagacctcgtcaatgttcatgctgccgggggcaaaaaaatgatgcaggcagctctcgaaggcttagaagaaggtacgccggctggaaaaaaacgtccgtcacttatcgcggtaacccagctgacaagcacatctgaacaaatcatgaaagatgaactgctgatcgaaaagtctctgattgatacggttgtgcactacagcaaacaggcggaagaaagcggactggatggagtggtctgctctgttcatgaagcaaaagccatttaccaagcggtgtcgccttcatttctgactgtcactccggggatcagaatgtcagaggacgctgcgaatgaccaagttcgcgtagcgacgcctgccattgcaagagagaaaggttcatcagcgattgtagtaggacgctcgattacaaaagcggaagacccggtaaaagcctataaggctgtcagacttgaatgggagggaatcaaatcttga

SEQ ID NO: 7 - protein, 30 aa, synthetic construct
LIGSDGGSGGGSTSRDHMVLHEYVNAAGIT